(19) United States
(12) Patent Application Publication
(10) Pub. No.: US 2014/0157474 A1
(43) Pub. Date: Jun. 12, 2014

(54) VENTILATED EYE SHIELD FOR SKI HELMET

(71) Applicants: Gerald F. ALESSI, Fairfield, CT (US); Gregg P. ALESSI, New York, NY (US)

(72) Inventors: Gerald F. ALESSI, Fairfield, CT (US); Gregg P. ALESSI, New York, NY (US)

(21) Appl. No.: 13/711,612

(22) Filed: Dec. 12, 2012

(51) Int. Cl. A61F 9/02

(52) U.S. Cl. CPC A61F 9/029; A61F 9/028
USPC 2/10

(57) ABSTRACT

The present subject matter is directed to a ventilated eye shield for a ski helmet. The eye shield comprises means of rotatably connecting the eye shield to the helmet body, the eye shield having raised and lowered positions relative to the helmet body, a straight upper edge, a lower edge having a notch adapted to rest on the skier's nose, a resilient foam separator attached to the lower inside edge of the face shield isolating the vision region from the breathing region and air inlets on either side of the lower edge of the face shield. The foam insert is contoured to prevent snow or debris from penetrating between the inside of the face shield and the skier's face. Outside air may penetrate from the air inlets on the sides of the eye shield, ventilate and prevent fogging of the inside surface of the eye shield.

### FIELD OF THE INVENTION

This invention relates to an improved eye shield for a ski helmet designed to reduce fogging, improve peripheral vision and prevent snow and debris from penetrating inside the helmet.

### BACKGROUND OF THE INVENTION

Ski helmets are generally designed to be worn with goggles. This is generally inconvenient for wearers of prescription glasses. In addition, wearing of goggles restricts peripheral vision and the goggles may become fogged from the differential in temperature between the outside and inside of the goggles. Furthermore, the open faced design of most helmets allow for snow, debris and cold air to penetrate inside the helmet and cover and chill the face of the user.

Early ski helmet designs such as taught by U.S. Pat. No. 4,287,615 to Morin, incorporate goggles or a shield which can be retracted under the shell of the helmet. Such designs do not prevent fogging or debris from penetrating the helmet.

Manufacturers have sought to overcome some of the limitations of open faced ski helmets with the provision of a fixed or retractable face shield. U.S. Pat. No. 7,716,754 to Ross teaches the use of a retractable face shield where no space exists between the top edge of the shield and the helmet in a closed position, partially controlling the airflow. Without sealing means to control airflow from the bottom of the shield, the shield cannot prevent snow and debris from entering via the bottom of the shield. Furthermore, the warm air from the breath of the wearer can penetrate the space below the face shield and fog the shield.

Various complex solutions have been provided to control fogging of an eye shield in cold weather. U.S. Pat. No. 6,425,143 to Benedict teaches a helmet having ventilation for fog management and respiration. The helmet has a shell shaped to define a cavity for a wearer's head. A transparent shield is engaged with the shell. The shell and shield cooperate to fully enclose the wearer's head. The shield has a moveable visor attached thereon. The helmet has at least one first air inlet and first air outlet. The shell and the inner surface of the shield cooperate to define a first airflow path that enables air to enter through the first air inlet, flow across the inner surface of the shield to draw off moisture from inner surface of the shield to control fogging, and exit through the first air outlet.

U.S. Pat. No. 5,694,650 to Hong teaches a heated double lens face shield with passive defogging. The face shield has a face shield frame, lower and upper face shield frame vents, and upper and lower air guides to direct the flow of air flowing through the upper and lower vents.

Other cold-weather helmet designs, for uses such as snowmobiling as taught by U.S. Pat. No. 6,795,979 to Fournier incorporate an eye shield pivotally connected to the helmet. An eye shield heating system on the eye shield electrically connects to the head portion of the helmet to provide electric power to the heating system. Such a design would be overly costly, cumbersome and heavy for recreational skiers.

There is therefore a need for a low cost retractable eye shield for ski helmets which will reduce fogging, improve peripheral vision and protect the face from snow and debris.

There is also a need for an eye shield which can be worn with prescription glasses.

### SUMMARY OF THE INVENTION

The disclosure teaches a retractable eye shield for a ski helmet comprising a separator to isolate the vision region to prevent snow and debris from penetrating behind the shield.

One aspect of the disclosure is to prevent warm air from the wearer's breath to penetrate behind the eye shield and fog the interior surface of the eye shield.

Another aspect of the disclosure is to provide airflow for ventilation of the eye shield and prevent fogging of the interior surface of the eye shield.

A further aspect of the invention is to provide maximum peripheral vision.

Another further aspect of the invention is to allow the skier to wear prescription glasses.

The ski helmet consists of a helmet body and a retractable eye shield. The retractable eye shield of the current invention is pivotally connected to the helmet body and can be rotated up and down between a raised and lowered position. The eye shield is curved to a crescent shape with a radius substantially matching the radius of the helmet body.

In a preferred embodiment, the eye shield is attached to the body helmet by two fasteners on either side of the helmet body. As the eye shield rises, it rotates upward, sliding over the top of the helmet shell, substantially out of the wearer's field of vision. In the lowered position the eye shield is positioned in front of the wearer's eyes and a notch in the lower edge of the eye shield rests against the bridge of the nose of the wearer. The lower edge of the eye shield is bordered with a separator made from resilient foam material sealing the eye shield against the face of the wearer. In the lowered position, substantially no space exists between the bottom of the eye shield and the face of the wearer for debris or snow to enter. The separator also prevents warm air from the wearer's breath to penetrate inside the eye shield. In order to prevent fogging of the interior surface of the eye shield, outside cold ventilation air is allowed to enter from lower openings in the eye shield. Air may only enter from the left and right sides of the eye shield, in the opening between the end of the foam insert and the helmet shell. The airflow will be low enough so as not to cool the face of the skier but sufficient to prevent fogging of the inside of the eye shield. Because the area above the skier's mouth and nose is sealed from the outside by the foam insert, the wearer's breath cannot enter the eye shield and cause the eye shield to fog.

The eye shield is also positioned sufficiently away from the user's face so as to permit the user to wear prescription glasses if needed. This offers maximum peripheral vision to the user.

Because of the frictional engagement of the two fasteners holding the visor to the helmet body, the skier can also adjust the position of the eye shield to any position between the fully closed and open positions.

The eye shield controls the movement of air around and behind the eye shield. In the fully closed position, substantially all the outside air is prevented from penetrating the vision region. Air inlets located on the left and right sides of the lower edge of the eye shield allow a limited amount of cold outside air to penetrate behind the eye shield, mix with the warmer air behind the eye shield and prevent the eye shield from fogging. An opening between the top of the eye shield and the helmet can also be provided allow the outside air to exit.

The ventilated eye shield for a ski helmet comprises:
a) means of rotatably connecting the eye shield to the helmet body, the eye shield being continuously settable between a raised position and a lowered position relative to the helmet body wherein the eye shield is adapted to be in front of the wearer's eyes when set in the lowered position and is adapted to be substantially out of the wearer's field of vision when in the raised position;
b) an upper edge and a lower edge, said lower edge having a notch adapted to rest on the bridge of the wearer's nose when the eye shield is set in the lowered position;
c) a separator bordering the wearer facing side of the lower edge of the eye shield wherein the separator isolates the vision region from the breathing region of the wearer when the eye shield is set in the lowered position and wherein the separator is contoured to prevent snow or debris from penetrating the vision region when the eye shield is set in the lowered position;
d) air inlets on the left and right sides of the lower edge of the eye shield, said air inlets defining a passage for cold outside air to penetrate behind the eye shield, wherein said outside air prevents fogging of the eye shield.

### BRIEF DESCRIPTION OF THE DRAWINGS

FIG. 1 is a perspective view of the disclosed eye shield attached to a ski helmet in the lowered position.

FIG. 2 is a perspective view of the disclosed eye shield detached from the helmet shell showing the foam insert and rubber trim along the lower edge of the eye shield.

FIG. 3 is a perspective view of the disclosed eye shield attached to a ski helmet in the lowered position as worn by a wearer with prescription glasses.

FIG. 4 is a perspective view of the disclosed eye shield attached to a ski helmet as worn by a wearer in the fully raised position.

FIG. 5 is a top view of the foam strip attached to the lower edge of the disclosed eye shield.

### DETAILED DESCRIPTION OF THE INVENTION

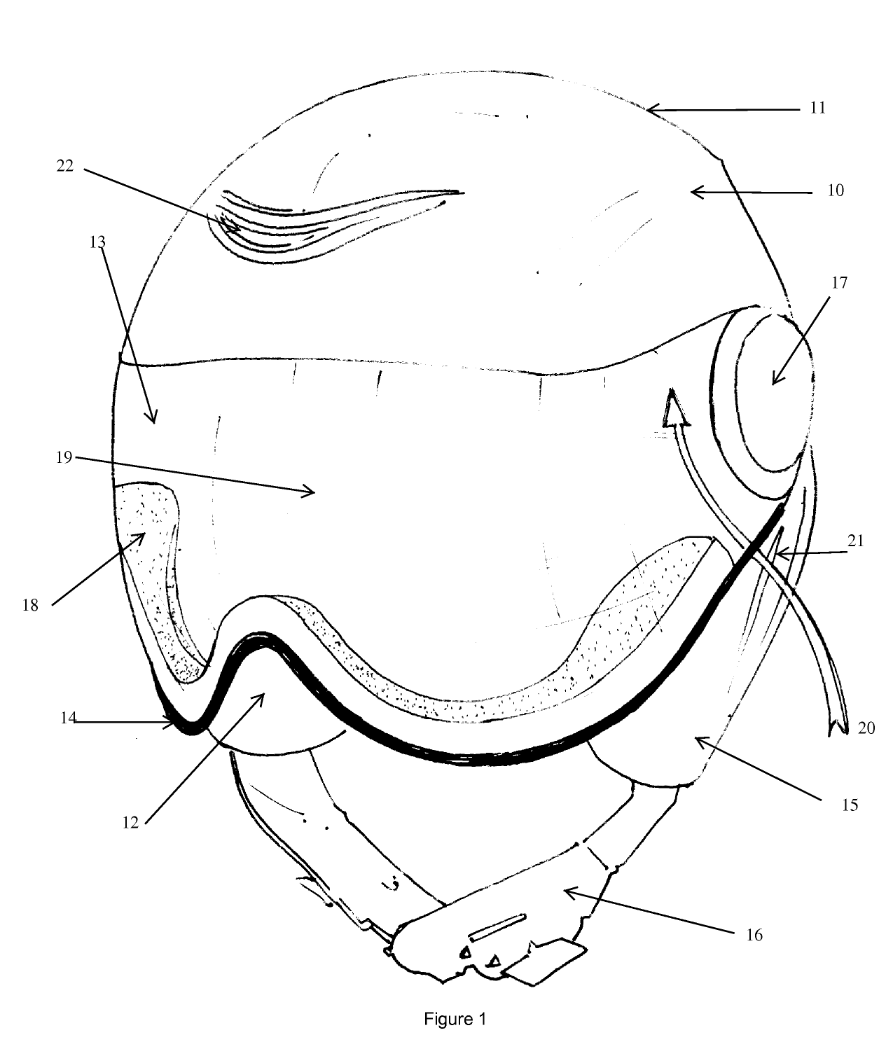

As illustrated in FIG. 1, the helmet 10 comprises a shell 11. The shell 11 defines a cavity 12 therein. The shell 11 is adapted to substantially enclose the head of a person wearing the helmet 10, when the wearer's head is placed within the cavity 12. The shell 11 may comprise any suitably durable material, including but not limited to metal, solid or foamed plastic, and fiberglass. The shell 11 may comprise multiple layers of material, such as a rigid outer layer and a padded, shock absorbing inner layer. The shell 11 may be formed as a single piece, or may comprise a plurality of pieces secured together. Referring to FIG. 1 of the drawings, a crescent-shaped eye shield 13 is rotatably connected to the shell in accordance with an embodiment of the present invention. Its periphery is continuous and devoid of sharp corners. The eye shield 13 has substantially the same radius of curvature as the shell 11. It is bordered in its lower part by a rubber trim 14 which provides additional nose and face protection to the wearer. In a preferred embodiment, the eye shield is made from a high impact resistant clear or color coated, UV absorbing, thermoplastic resin.

In a preferred embodiment, the helmet 10 also includes an ear protection portion 15 and adjustable chinstrap 16. As can be seen in FIG. 1, the eye shield 13 is pivotally connected to helmet 10 at right and left pivot locations 17 which are preferably fasteners with frictional engagement so as to allow multiple settings between the raised and lowered positions. The lower edge of the eye shield 13 is contoured to wrap around the nose and cover the upper portion of the face of the wearer when in fully lowered position.

Advantageously, the eye shield comprises a separator 18 between the vision region 19 and the breathing region 20. In a preferred embodiment, the separator comprises a foam strip preferably attached to the wearer facing side of the lower edge of the eye shield with an adhesive material. The separator follows the lower contour of the eye shield except for approximately two inches at either side of the shield. The eye shield can be retracted upward or downward about the screws 16. In the fully lowered or closed position shown in FIG. 1, the lower edge of the eye shield rests against the bridge of the nose of the wearer and the foam strip 18 provides a tight fit of the shield to the face. In the fully lowered position, cold air 20 can only penetrate inside the eye shield through left and right side lower edge openings 21. Optional top openings 22 in the shell may permit air to exit. When the eye shield is in the lowered position, separator 18 seals the space around the vision region 19 and prevents the wearer's breath to mix with the cold ambient air and fog the eye shield.

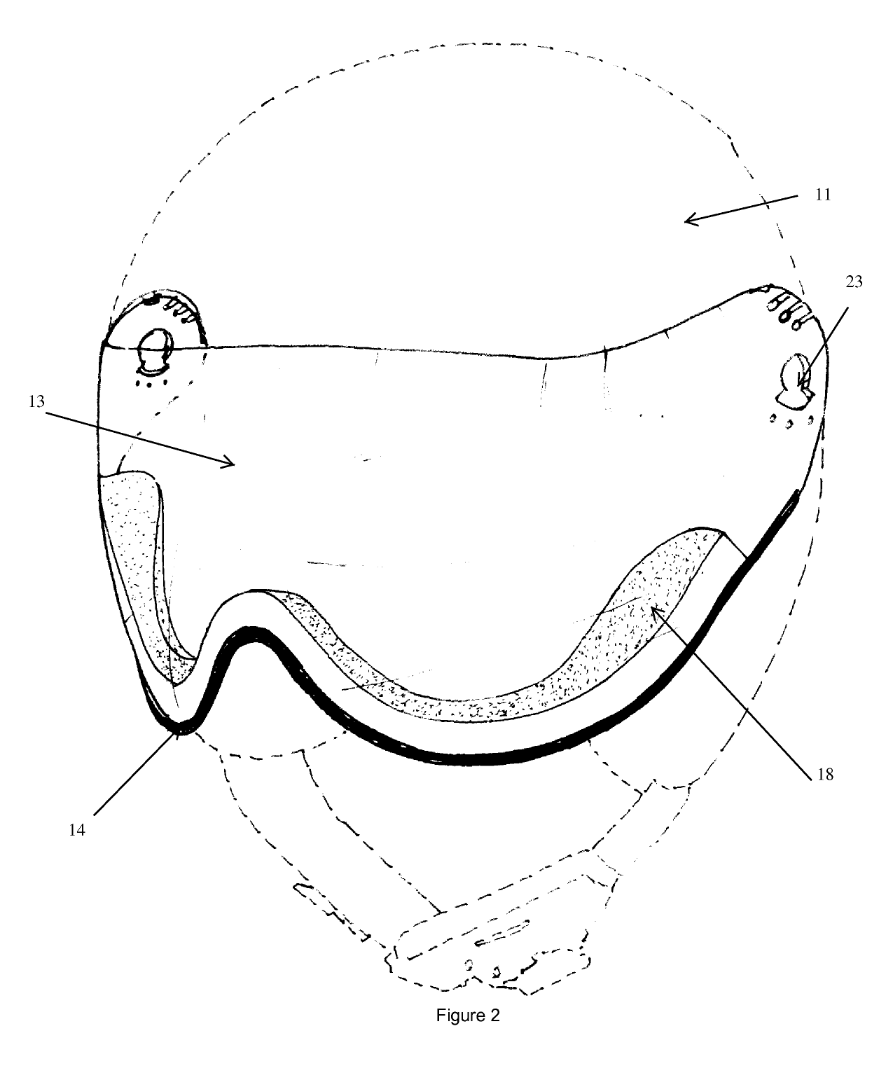

FIG. 2 is a perspective view of a preferred embodiment of the eye shield 13 detached from the helmet shell 11. The foam insert 18 is approximately 0.5″ thick and with a depth varying from approximately 1″ at the edges to 0.5″ at the center of the eye shield. The rubber trim 14 is approximately 0.25″ high and warps around the bottom edge of the eye shield 13. Openings 23 in the sides of the eye shield are connected to the pivot locations 17 on the helmet shell.

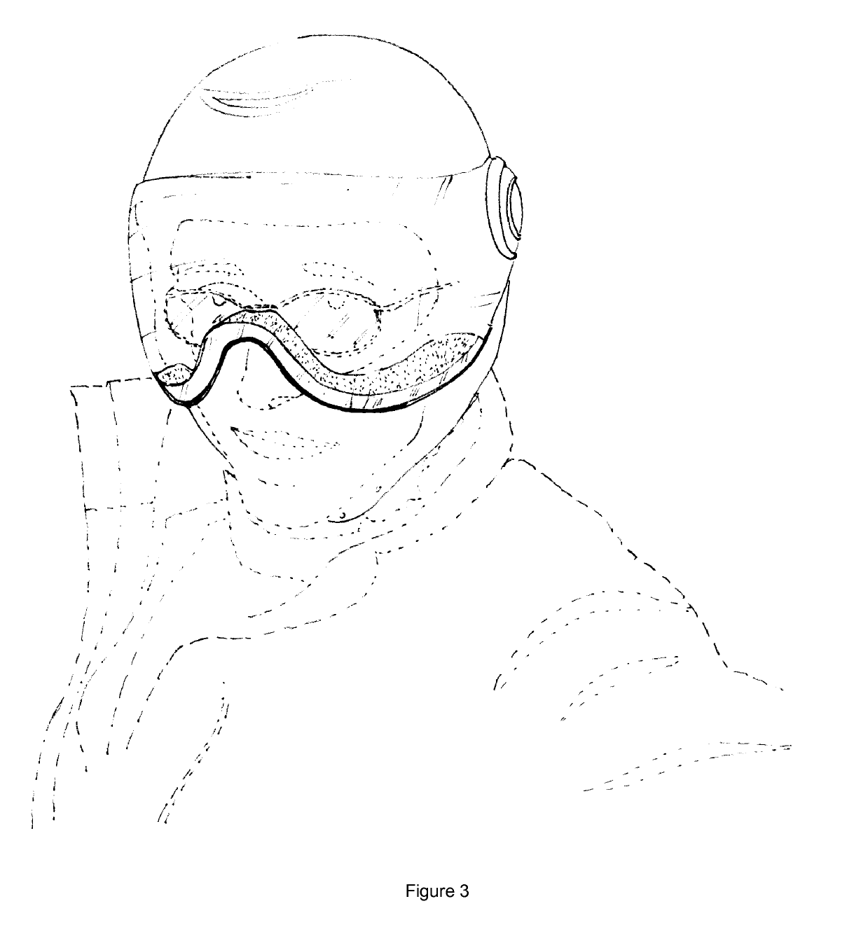

FIG. 3 is a drawing of a preferred embodiment of the eye shield attached to the ski helmet as worn by a wearer. The helmet is shown in the fully lowered position with the rubber trim along the lower edge of the eye shield resting against the nose of the skier. It is to be clearly understood that the foam strip provides a tight seal against the face of the skier, preventing snow or debris from entering from the lower edge of the eye shield. It is also clearly shown that the eye shield is compatible with the skier wearing prescription glasses or goggles even in the fully downward position.

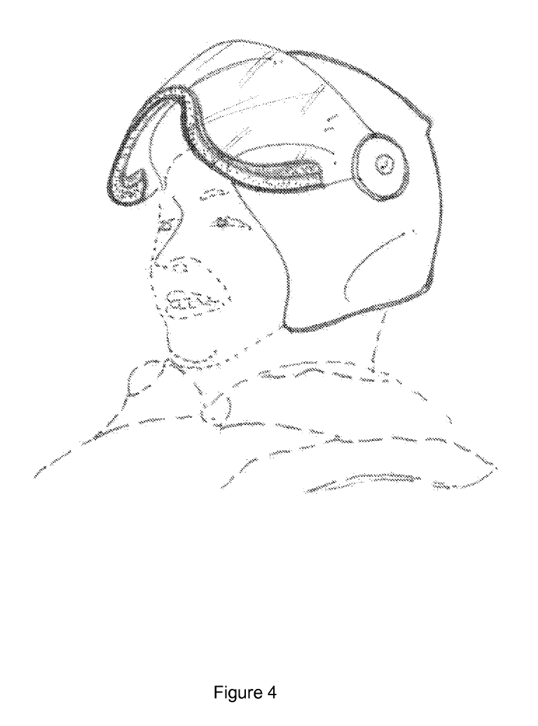

FIG. 4 is a drawing of a preferred embodiment of the eye shield attached to the ski helmet as worn by a wearer in the fully raised position.

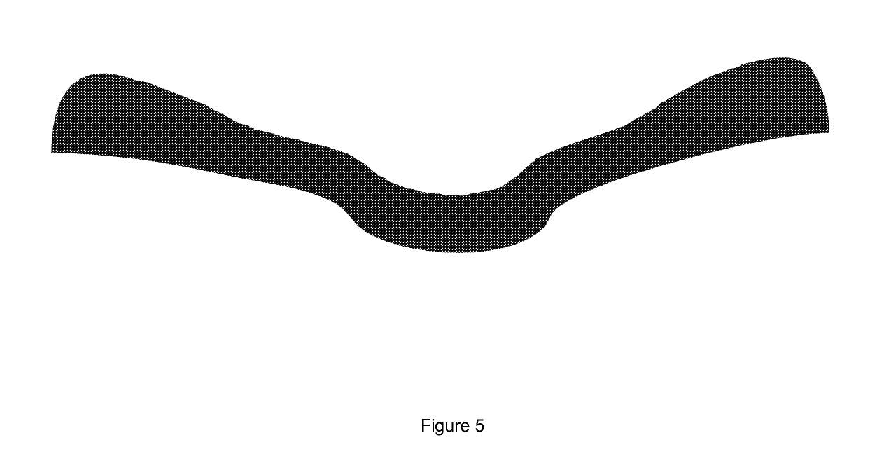

FIG. 5 is a drawing of a preferred embodiment of the foam insert 18 attached to the lower edge of the eye shield.

What is claimed is:

1. An eye shield for a ski helmet protecting a wearer's vision region comprising:
a) means of rotatably connecting the eye shield to the helmet body, the eye shield being continuously settable between a raised position and a lowered position relative to the helmet body wherein the eye shield is adapted to be in front of the wearer's eyes when set in the lowered position and is adapted to be substantially out of the wearer's field of vision when in the raised position;
b) an upper edge and a lower edge, said lower edge having a notch adapted to rest on the bridge of the wearer's nose when the eye shield is set in the lowered position;
c) a separator bordering the wearer facing side of the lower edge of the eye shield wherein the separator isolates the vision region from the breathing region of the wearer when the eye shield is set in the lowered position and wherein the separator is contoured to prevent snow or debris from penetrating the vision region when the eye shield is set in the lowered position;

2. The eye shield of claim 1 wherein the means of rotatably connecting the eye shield to the helmet shell comprise fasteners with frictional engagement so as to allow multiple eye shield settings between the raised and lowered positions.

3. The eye shield of claim 1 wherein the eye shield is made from a high impact resistant clear or color coated, UV absorbing, thermoplastic resin.

4. The eye shield of claim 1 wherein the eye shield is curved to a crescent shape with a radius substantially matching the radius of the helmet body.

5. The eye shield of claim 1 wherein the separator is a resilient foam insert is approximately 0.5″ thick and with a depth varying from approximately 1″ at the edges to 0.5″ at the center of the eye shield.

6. The eye shield of claim 1 wherein the lower edge of the eye shield is bordered by a rubber trim.

7. An eye shield for a ski helmet protecting a wearer's vision region comprising:
a) means of rotatably connecting the eye shield to the helmet body, the eye shield being continuously settable between a raised position and a lowered position relative to the helmet body wherein the eye shield is adapted to be in front of the wearer's eyes when set in the lowered position and is adapted to be substantially out of the wearer's field of vision when in the raised position;
b) an upper edge and a lower edge, said lower edge having a notch adapted to rest on the bridge of the wearer's nose;
c) air inlets on the left and right sides of the lower edge of the eye shield, said air inlets defining a passage for cold outside air to penetrate behind the eye shield, wherein said outside air prevents fogging of the eye shield.

8. The eye shield of claim 7 comprising a top opening for the outside air to exit.
